SEQUENCE LISTING (6 sequences)

SEQ ID NO: 1 - DNA, 1593 bp, Homo sapiens
gcggtgcccttgcggcgcagctggggtcgcggccctgctccccgcgctttcttaaggcccgcgggcggcgcaggagcggcactcgtggctgtggtggcttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgagtatgttaagctctttatgactgtttttgtagtggtatagagtactgcagaatacagtaagctgctctattgtagcatttcttgatgttgcttagtcacttatttcataaacaacttaatgttctgaataatttcttactaaacattttgttattgggcaagtgattgaaaatagtaaatgctttgtgtgattgaatctgattggacattttcttcagagagctaaattacaattgtcatttataaaaccatcaaaaatattccatccatatactttggggacttgtagggatgcctttctagtcctattctattgcagttatagaaaatctagtcttttgccccagttacttaaaaataaaatattaacactttcccaagggaaacactcggctttctatagaaaattgcactttttgtcgagtaatcctctgcagtgatacttctggtagatgtcacccagtggtttttgttaggtcaaatgttcctgtatagtttttgcaaatagagctgtatactgtttaaatgtagcaggtgaactgaactggggtttgctcacctgcacagtaaaggcaaacttcaacagcaaaactgcaaaaaggtggtttttgcagtaggagaaaggaggatgtttatttgcagggcgccaagcaaggagaattgggcagctcatgcttgagacccaatctccatgatgacctacaagctagagtatttaaaggcagtggtaaatttcaggaaagcagaagtt

SEQ ID NO: 2 - protein, 222 aa, Homo sapiens
MLSRAVCGTSRQLAPVLGYLGSRQKHSLPDLPYDYGALEPHINAQIMQLHHSKHHAAYVNNLNVTEEKYQEALAKGDVTAQIALQPALKFNGGGHINHSIFWTNLSPNGGGEPKGELLEAIKRDFGSFDKFKEKLTAASVGVQGSGWGWLGFNKERGHLQIAACPNQDPLQGTTGLIPLLGIDVWEHAYYLQYKNVRPDYLKAIWNVINWENVTERYMACKK

SEQ ID NO: 3 - DNA, 21 bp, Artificial Sequence
  Synthetic oligonucleotide
caacggatttggtcgtattgg

SEQ ID NO: 4 - DNA, 25 bp, Artificial Sequence
  Synthetic oligonucleotide
tgatggcaacaatatccactttacc

SEQ ID NO: 5 - DNA, 23 bp, Artificial Sequence
  Synthetic oligonucleotide
gttcaatggtggtggtcatatca

SEQ ID NO: 6 - DNA, 20 bp, Artificial Sequence
  Synthetic oligonucleotide
gcaactcccctttgggttct

(19) United States
(12) Patent Application Publication
(10) Pub. No.: US 2016/0244832 A1
(43) Pub. Date: Aug. 25, 2016

(54) METHODS AND KITS FOR DIAGNOSING, PROGNOSING AND MONITORING PARKINSON'S DISEASE

(71) Applicant: Rosalind Franklin University of Medicine and Science, North Chicago, IL (US)

(72) Inventors: Judith Ann POTASHKIN, North Chicago, IL (US); Jose Alfredo SANTIAGO, North Chicago, IL (US)

(73) Assignee: Rosalind Franklin University of Medicine and Science, North Chicago, IL (US)

(21) Appl. No.: 14/870,931

(22) Filed: Sep. 30, 2015

Related U.S. Application Data
(60) Provisional application No. 62/120,842, filed on Feb. 25, 2015.

(51) Int. Cl. C12Q 1/68

(52) U.S. Cl. CPC C12Q 1/6883; C12Q 2600/118; C12Q 2600/158; C12Q 2600/112

(57) ABSTRACT

Increasing evidence indicates that Parkinson's disease (PD) and type 2 diabetes (T2DM) share dysregulated molecular networks. 84 genes shared between PD and T2DM were identified from curated disease-gene databases. Nitric oxide biosynthesis, lipid and carbohydrate metabolism, insulin secretion and inflammation were identified as common dysregulated pathways. A network prioritization approach was implemented to rank genes according to their distance to seed genes and their involvement in common biological pathways. This disclosure reinforces the idea that shared molecular networks between PD and T2DM provide an additional source of biologically meaningful biomarkers.

### CROSS REFERENCE

This application is related to U.S. provisional patent application No. 62/120,842, filed Feb. 25, 2015, the disclosure of which is incorporated by reference herein in its entirety. The sequence listing submitted herewith is incorporated by reference in its entirety.

### STATEMENT REGARDING FEDERALLY SPONSORED RESEARCH

This invention was made with U.S. government support by the U.S. Army Medical Research and Material Command under award numbers W81XWH-09-0708 and W81XWH13-1-0025. The U.S. Government has certain rights in the invention.

### BACKGROUND OF THE DISCLOSURE

Parkinson's disease (PD; also known as idiopathic or primary parkinsonism, hypokinetic rigid syndrome (HRS), or paralysis agitans) belongs to a group of conditions called motor system disorders, which are the result of the loss of dopamine-producing brain cells. The four primary symptoms of PD are tremor, or trembling in hands, arms, legs, jaw, and face; rigidity, or stiffness of the limbs and trunk; bradykinesia, or slowness of movement; and postural instability, or impaired balance and coordination. As these symptoms become more pronounced, patients may have difficulty walking, talking, or completing other simple tasks. PD usually affects people over the age of 60. Early symptoms of PD are subtle and occur gradually. In some people the disease progresses more quickly than in others. As the disease progresses, the shaking, or tremor, which affects the majority of people with PD may begin to interfere with daily activities. Other symptoms may include depression and other emotional changes; difficulty in swallowing, chewing, and speaking; urinary problems or constipation; skin problems; and sleep disruptions. There are currently no blood or laboratory tests that have been proven to help in diagnosing sporadic PD. Therefore the diagnosis is based on medical history and a neurological examination, but the disease can be difficult to diagnose accurately. Doctors may sometimes request brain scans or laboratory tests in order to rule out other diseases.

At present, there is no cure for PD, but a variety of medications provide dramatic relief from the symptoms. Usually, affected individuals are given levodopa (L-DOPA; SINEMET™, PARCOPA™, ATAMET™, STALEVO™, MADOPAR™, and PROLOPA™) combined with carbidopa (LODOSYN™) (products containing a combination of levodopa and carbidopa include DUOPA® and RYTARY®). Carbidopa delays the conversion of levodopa into dopamine until it reaches the brain. Nerve cells can use levodopa to make dopamine and replenish the brain's dwindling supply. Although levodopa helps at least three-quarters of PD cases, not all symptoms respond equally to the drug. Bradykinesia and rigidity typically respond best, while tremor may be only marginally reduced. Problems with balance and other symptoms may not be alleviated at all. Anticholinergics may help control tremor and rigidity. Dopamine agonists, such as bromocriptine, pergolide, pramipexole, ropinirole, piribedil, cabergoline, apomorphine and lisuride, mimic the role of dopamine in the brain, causing the neurons to react as they would to dopamine. An antiviral drug, amantadine, also appears to reduce symptoms. In May 2006, the FDA approved rasagiline to be used along with levodopa for patients with advanced PD or as a single-drug treatment for early PD.

In some cases, surgery may be appropriate if the disease does not respond to drugs. A therapy called deep brain stimulation (DBS) has now been approved by the U.S. Food and Drug Administration. In DBS, electrodes are implanted into the brain and connected to a small electrical device called a pulse generator that can be externally programmed. DBS can reduce the need for levodopa and related drugs, which in turn decreases the involuntary movements called dyskinesias that are a common side effect of levodopa. It also helps to alleviate fluctuations of symptoms and to reduce tremors, slowness of movements, and gait problems. DBS requires careful programming of the stimulator device in order to work correctly.

This disclosure demonstrates that integration of shared molecular networks provides a useful framework to prioritize candidate biomarkers in a biologically relevant context, and that expression of highly ranked genes identified within shared dysregulated pathways can be used as diagnostic biomarkers for PD.

### SUMMARY OF THE DISCLOSURE

There is a need in the art for a better understanding of the underlying disease mechanism and methods to facilitate the discovery of accurate biomarkers and therapeutic targets for Parkinson's disease. This disclosure demonstrates that integration of shared molecular networks provides a useful framework to prioritize candidate biomarkers in a biologically relevant context, and that expression of highly ranked genes identified within shared dysregulated pathways can be used as diagnostic biomarkers for PD.

In one aspect, the disclosure provides a method for diagnosing, prognosing or monitoring Parkinson's Disease (PD) in a human subject, the method comprising: (a) obtaining a blood sample from a human subject suspected of having PD; (b) determining the expression level of at least one gene in the blood sample from the human subject suspected of having PD, wherein the at least one gene is selected from: SOD2, MT-ND1, TNF, IFNG, TP53, IL6, AKT1, HNF4A, HMOX1, FAS, APP, CYP17A1, IGF1, PTGS2, SOD1, BDNF, NOS2, TGM2, GCH1, UCHL1, IL1B, HNF1A, APOE, IGF2, CYP1A1, PPARG, SLC18A2, CD14, PINK1, INS, PARP1, NFKB1, SLC2A4, IDE, DRD2, GAD2, SORBS1, CP, TH, TSC2, PON1, E2F1, CXCR4, CDKN2A, KCNJ2, PPARGC1A, HGF, OPRM1, TF, ACE, CADM1, NQO1, GAD1, GH1, HFE, CXCL12, ABCB1, MAOB, BTG1, ABCC8, PDX1, ADH1C, CCL5, TCF7L2, ATF6, GPX1, CCL2, VDR, MTHFR, IL8, KIF11, MMP16, GSTM1, HP, NPPB, SEMA6A, NCAM2, SERPINB1, NAT2, MBNL1, PCDH18, OLFM4, RBMS3 and TBC1D22A; and (c) comparing the expression level of the at least one gene expressed in the blood sample to the expression level of the at least one gene in a non-PD, healthy control sample, whereby the increased or decreased expression level of the at least one gene expressed in the blood sample from the human subject suspected of having PD as compared to the non-PD sample is indicative of PD, thereby diagnosing the human subject as having PD.

In another aspect, the disclosure provides a method of treating a human subject for Parkinson's Disease (PD), the method comprising: (a) obtaining a diagnosis identifying a human subject as having PD, wherein the diagnosis was obtained by: (i) obtaining a blood sample from a human subject suspected of having PD; (ii) determining the expression level of at least one gene in the blood sample from the human subject suspected of having PD, wherein the at least one gene is selected from: SOD2, MT-ND1, TNF, IFNG, TP53, IL6, AKT1, HNF4A, HMOX1, FAS, APP, CYP17A1, IGF1, PTGS2, SOD1, BDNF, NOS2, TGM2, GCH1, UCHL1, IL1B, HNF1A, APOE, IGF2, CYP1A1, PPARG, SLC18A2, CD14, PINK1, INS, PARP1, NFKB1, SLC2A4, IDE, DRD2, GAD2, SORBS1, CP, TH, TSC2, PON1, E2F1, CXCR4, CDKN2A, KCNJ2, PPARGC1A, HGF, OPRM1, TF, ACE, CADM1, NQO1, GAD1, GH1, HFE, CXCL12, ABCB1, MAOB, BTG1, ABCC8, PDX1, ADH1C, CCL5, TCF7L2, ATF6, GPX1, CCL2, VDR, MTHFR, IL8, KIF11, MMP16, GSTM1, HP, NPPB, SEMA6A, NCAM2, SERPINB1, NAT2, MBNL1, PCDH18, OLFM4, RBMS3 and TBC1D22A; and (iii) comparing the expression level of the at least one gene expressed in the blood sample to the expression level of the at least one gene expressed in a non-PD, healthy control sample, whereby the increased or decreased expression level of the at least one gene expressed in the blood sample from the human subject suspected of having PD as compared to the non-PD sample is indicative of PD, thereby diagnosing the human subject as having PD; and (b) administering to the subject a PD treatment regimen.

In yet another aspect, the disclosure provides, a Parkinson's Disease diagnosis, prognosis or monitoring kit, consisting of a set of probes suitable for the detection and quantification of the nucleic acid expression of at least one gene selected from: SOD2, MTND1, TNF, IFNG, TP53, IL6, AKT1, HNF4A, HMOX1, FAS, APP, CYP17A1, IGF1, PTGS2, SOD1, BDNF, NOS2, TGM2, GCH1, UCHL1, IL1B, HNF1A, APOE, IGF2, CYP1A1, PPARG, SLC18A2, CD14, PINK1, INS, PARP1, NFKB1, SLC2A4, IDE, DRD2, GAD2, SORBS1, CP, TH, TSC2, PON1, E2F1, CXCR4, CDKN2A, KCNJ2, PPARGC1A, HGF, OPRM1, TF, ACE, CADM1, NQO1, GAD1, GH1, HFE, CXCL12, ABCB1, MAOB, BTG1, ABCC8, PDX1, ADH1C, CCL5, TCF7L2, ATF6, GPX1, CCL2, VDR, MTHFR, IL8, KIF11, MMP16, GSTM1, HP, NPPB, SEMA6A, NCAM2, SERPINB1, NAT2, MBNL1, PCDH18, OLFM4, RBMS3 and TBC1D22A.

These and other features and advantages of the present disclosure will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

FIG. 1 illustrates the integrative network approach. Well-characterized genes associated with PD (purple circles) and type 2 diabetes mellitus (T2DM) (magenta circles) were mapped to the functional linkage network (FLN) and specified as training set. Shared genes between PD and T2DM (yellow circles) were collected from multiple databases and mapped to the human FLN (black). A random walk algorithm with restart (RWR) was implemented to prioritize the list of shared genes between PD and T2DM according to their distance to known disease genes and in terms of biological pathways involved. A highly ranked gene was evaluated as diagnostic biomarker for PD on RNA samples from whole blood obtained from two independent clinical trials.

FIG. 2 illustrates a biological functional analysis of candidate genes. Network of interactions among PD and T2DM shared genes, as retrieved by GeneMANIA. Shared genes between PD and T2DM are displayed in yellow circles and other genes with the greatest number of interactions with shared genes are displayed in gray circles. The size of the gray nodes represents the degree of association with the input genes (i.e., smaller size represents low connectivity). The most represented pathways retrieved by GeneMANIA are displayed using GO annotations and Q-values of significance.

FIGS. 3A, 3B and 3C show the evaluation of SOD2 as a potential biomarker for PD. FIG. 3A shows the relative abundance of SOD2 mRNA in blood of PD patients (black circles) compared to healthy controls (white circles) in samples from the HBS cohort. FIG. 3B. shows the replication of biomarker expression in an independent set of samples from patients enrolled in the PROBE study. Relative abundance of each biomarker was calculated using GAPDH as a reference gene and healthy controls as calibrator. Error bars represent standard error. FIG. 3C. shows the ROC curve analysis to evaluate the diagnostic accuracy of SOD2.

FIGS. 4A, 4B, 4C, 4D, 4E and 4F show the validation of each prioritization step. The performance of each prioritization step was validated by computing values for receiver operating curve (ROC) and area under the curve (AUC) through the leave-one-out validation method using GPEC.

### DETAILED DESCRIPTION OF THE DISCLOSURE

The present disclosure relates to methods of integrating of shared molecular networks to provide a useful framework to prioritize candidate biomarkers in a biologically relevant context, and demonstrates that expression of highly ranked gene biomarkers identified within shared dysregulated pathways can be used in diagnosing, prognosing or monitoring Parkinson's disease.

Accumulating epidemiological evidence suggests a risk of Parkinson's disease (PD) among patients with type 2 diabetes mellitus (T2DM). However, there remains conflict among some studies. For example, several groups suggest an inverse association between PD and T2DM and other studies have not found a significant association. Despite this discrepancy, T2DM is associated with more severe symptoms in PD. T2DM contributes to postural instability and gait difficulty in PD and insulin resistance is associated with an increased risk of dementia in PD. Besides insulin resistance, dysregulation in other shared biological pathways including mitochondrial dysfunction, endoplasmic reticulum (ER) stress and inflammation may be a plausible explanation for the coexistence of both aging diseases.

Both PD and T2DM are considered idiopathic diseases in which a combination of genetic and environmental factors are likely to be involved in the disease pathogenesis. In fact, genetic risk factors identified by genome-wide association studies (GWAS) accounts for approximately 5-10% of the PD and T2DM cases. Several system-biology approaches including animal models and network analysis have been used to understand the molecular mechanisms underlying the linkage between PD and T2DM. For example, diabetic mice treated with 1-methyl-4-phenyl-1,2,3,6-tetrahydropyridine (MPTP) displayed an exacerbated neurodegeneration accompanied by inflammation and ER-stress. In parallel, an integrative network-based approach restricted to data from only GWAS was used to investigate the potential molecular framework linking PD and T2DM and to identify potential biomarkers with clinical applicability. Results from these studies identified the amyloid precursor protein (APP) mRNA as a biomarker for PD. Similarly, a network approach identified PTPN1 mRNA as a diagnostic biomarker in progressive supranuclear palsy, an atypical parkinsonian disorder sometimes misdiagnosed as PD.

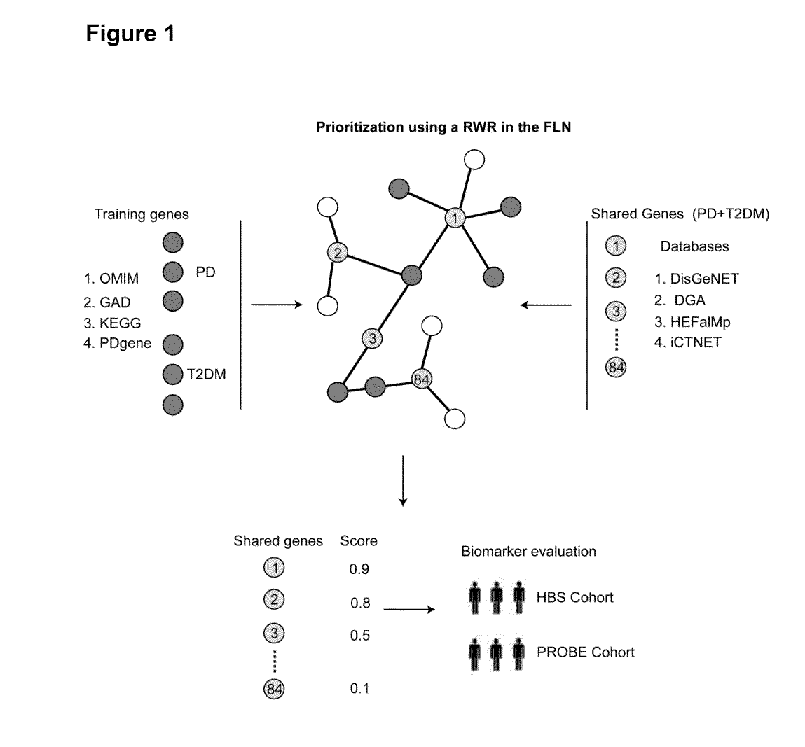

Network analysis was used to integrate data from publicly available and curated disease-gene databases to further investigate the connection between both diseases. The disease-gene associations derived from the databases included in this disclosure are not strictly determined by GWAS, thus allowing the exploration of other potentially interesting genes that may have a more modest association. Because PD and T2DM are sporadic and environmental factors play a role in disease etiology and development, it is important to use databases that include genetic disease associations identified through studies that tested environmental factors such as toxin. The systematic network approach of this disclosure is illustrated in FIG. 1. Briefly, genes shared between PD and T2DM were collected from several databases and mapped into the human functional linkage network (FLN). A random walk algorithm with restart (RWR) was implemented to rank the group of genes shared between PD and T2DM. The applicability of the network prioritization approach was evaluated by testing the most highly ranked gene as a potential diagnostic biomarker for PD. This disclosure relates to potential mRNA biomarkers from blood samples that can be used for diagnosing, prognosing or monitoring patients with PD.

It should be understood that the embodiments described herein, are provided for explanatory purposes, and are not intended to be limiting.

All publications, patents and patent applications cited herein are hereby expressly incorporated by reference for all purposes to the extent they are consistent with this disclosure.

Methods well known to those skilled in the art can be used to construct expression vectors and recombinant bacterial cells according to this disclosure. These methods include in vitro recombinant DNA techniques, synthetic techniques, in vivo recombination techniques, and PCR techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

In one aspect, the disclosure provides a method for diagnosing, prognosing or monitoring Parkinson's Disease (PD) in a human subject, the method comprising: (a) obtaining a blood sample from a human subject suspected of having PD; (b) determining the expression level of at least one gene in the blood sample from the human subject suspected of having PD, wherein the at least one gene is selected from: SOD2, MT-ND1, TNF, IFNG, TP53, IL6, AKT1, HNF4A, HMOX1, FAS, APP, CYP17A1, IGF1, PTGS2, SOD1, BDNF, NOS2, TGM2, GCH1, UCHL1, IL1B, HNF1A, APOE, IGF2, CYP1A1, PPARG, SLC18A2, CD14, PINK1, INS, PARP1, NFKB1, SLC2A4, IDE, DRD2, GAD2, SORBS1, CP, TH, TSC2, PON1, E2F1, CXCR4, CDKN2A, KCNJ2, PPARGC1A, HGF, OPRM1, TF, ACE, CADM1, NQO1, GAD1, GH1, HFE, CXCL12, ABCB1, MAOB, BTG1, ABCC8, PDX1, ADH1C, CCL5, TCF7L2, ATF6, GPX1, CCL2, VDR, MTHFR, IL8, KIF11, MMP16, GSTM1, HP, NPPB, SEMA6A, NCAM2, SERPINB1, NAT2, MBNL1, PCDH18, OLFM4, RBMS3 and TBC1D22A; and (c) comparing the expression level of the at least one gene expressed in the blood sample to the expression level of the at least one gene in a non-PD, healthy control sample, whereby the increased or decreased expression level of the at least one gene expressed in the blood sample from the human subject suspected of having PD as compared to the non-PD sample is indicative of PD, thereby diagnosing the human subject as having PD.

In an embodiment, the methods disclosed herein typically involve determining expression levels of at least one gene in a biological sample obtained from a human subject suspected of having PD. The methods may involve determining expression levels of at least 2, at least 3, at least 4, at least 5, at least 6, at least 7, at least 8, at least 9, at least 10, at least 20, at least 30, at least 40, at least 50, at least 60, at least 70, at least 80 genes in a biological sample obtained from an individual, wherein the genes are selected from: SOD2, MT-ND1, TNF, IFNG, TP53, IL6, AKT1, HNF4A, HMOX1, FAS, APP, CYP17A1, IGF1, PTGS2, SOD1, BDNF, NOS2, TGM2, GCH1, UCHL1, IL1B, HNF1A, APOE, IGF2, CYP1A1, PPARG, SLC18A2, CD14, PINK1, INS, PARP1, NFKB1, SLC2A4, IDE, DRD2, GAD2, SORBS1, CP, TH, TSC2, PON1, E2F1, CXCR4, CDKN2A, KCNJ2, PPARGC1A, HGF, OPRM1, TF, ACE, CADM1, NQO1, GAD1, GH1, HFE, CXCL12, ABCB1, MAOB, BTG1, ABCC8, PDX1, ADH1C, CCL5, TCF7L2, ATF6, GPX1, CCL2, VDR, MTHFR, IL8, KIF11, MMP16, GSTM1, HP, NPPB, SEMA6A, NCAM2, SERPINB1, NAT2, MBNL1, PCDH18, OLFM4, RBMS3 and TBC1D22A.

In one embodiment, the at least one gene is SOD2 encoded by SEQ ID NO: 01. Homo sapiens SOD2 superoxide dismutase 2, mitochondrial (Official Symbol: SOD2; Official Full Name: superoxide dismutase 2, mitochondrial; provided by HGNC). SOD2 is also known as: IPOB; MNSOD; or MVCD6. The accession number for the nucleic acid is >gi|67782304|ref|NM_000636.2| Homo sapiens superoxide dismutase 2, mitochondrial (SOD2), transcript variant 1, mRNA (SEQ ID NO: 01); the accession number for the protein is >gi|67782305|ref|NP_000627.2| Homo sapiens superoxide dismutase, mitochondrial isoform A precursor (SEQ ID NO: 02).

In another embodiment, the expression level is determined by detecting messenger RNA of the at least one gene. If mRNA is determined, then the method may further comprise reverse transcription of the messenger RNA prior to detecting.

In an embodiment, determining the expression level of the at least one gene is by measuring a level of fluorescence by a sequence detection system following a quantitative, real-time polymerase chain reaction (PCR) assay.

As used herein, a “sample” is a biological sample isolated from a subject and can include, but is not limited to, whole blood, serum, plasma, blood cells, endothelial cells, tissue biopsies, lymphatic fluid, ascites fluid, interstitial fluid, bone marrow, cerebrospinal fluid (CSF), saliva, mucous, sputum, sweat, urine, or any other secretion, excretion, or other bodily fluids. A “blood sample” refers to whole blood or any fraction thereof, including blood cells, serum and plasma.

As used herein, the terms “diagnosis”, “diagnostic”, “diagnosing”, refer to an identification of PD or to a predisposition of developing PD, based on a detection of at least one gene. The terms “prognosis”, “prognostic”, “prognosing”, refer to the ability of predicting, forecasting or correlating a given detection or measurement with a future outcome of PD in the patient (e.g., severity, likelihood of successfully treating, or survival). The disclosure also relates to monitoring the influence of agents, treatments or therapies for PD (e.g., drugs, compounds). As used herein, “monitoring” refers to determining the regression, progression, course and/or onset of, and/or prognoses of PD before any treatment or during treatment in order to assess the PD patient's improvement or lack thereof over time.

As used herein, the term “control sample” or “healthy control” refers to a sample from a subject that does not have PD or subject that does not have PD. In a particular embodiment, the control sample or healthy control does not have PD or is indicative of the absence of PD. Control samples can be obtained from patients/individuals not afflicted with PD. Other types of control samples may also be used. In a related facet, a control reaction may be designed to control the method itself (e.g., cell extraction, the capture, the amplification reaction or detection method, number of cells present in the sample, a combination thereof or any step which could be monitored to positively validate that the absence of a signal (e.g., the expression level of a gene) is not the result of a defect in one or more of the steps). Once a cut-off value is determined, a control sample giving a signal characteristic of the predetermined cut-off value can also be designed and used in the methods of the present invention. Diagnosis/prognosis tests are commonly characterized by the following 4 performance indicators: sensitivity (Se), specificity (Sp), positive predictive value (PPV), and negative predictive value (NPV).

As used herein, the terms “nucleic acid”, “polynucleotide”, “nucleotide”, and “oligonucleotide” can be used interchangeably to refer to single stranded or double stranded, nucleic acid comprising DNA, RNA, derivatives thereof, or combinations thereof.

Expression of the genes disclosed herein can be measured at the RNA level using any method known in the art. For example, expression can be measured using Real-Time PCR assays (RT-PCR), e.g., using primers specific for the differentially expressed sequences. As used herein, the term “real-time PCR” (also called quantitative real-time polymerase chain reaction) refers to a method for the detection and quantitation of an amplified PCR product based on incorporation of a fluorescent reporter dye; the fluorescent signal increases in direct proportion to the amount of PCR product produced and is monitored at each cycle, ‘in real-time’, such that the time point at which the first significant increase in the amount of PCR product correlates with the initial amount of target template. In one embodiment, real-time PCR can be preceded by reverse-transcription of the messenger RNA. RNA can also be quantified using, for example, other target amplification methods (e.g., TMA, SDA, NASBA), or signal amplification methods (e.g., bDNA). Alternatively, northern hybridization analysis using probes which specifically recognize one or more of the sequences or each gene can be used to determine gene expression.

The difference in the level of biomarker between normal and abnormal is preferably statistically significant and may be an increase in biomarker expression level or a decrease in biomarker expression level, and without any limitation of the method, achieving statistical significance, and thus the preferred analytical and clinical accuracy, generally but not always requires that combinations of several biomarkers be used together in panels and combined with mathematical algorithms in order to achieve a statistically significant biomarker index.

As used herein, the term “primer set” or “primers” refers to a pair of PCR primers that include a forward primer and reverse primer used in a PCR reaction and allows the generation of an amplicon. Numerous primers used in the context of the present disclosure can be readily determined by a person of ordinary skill in the art to which the present invention pertains. Non-limiting examples of primers are shown in SEQ ID NO: 3-6. A person skilled in the art can design numerous other primers based on the teachings herein and the common general knowledge. As used herein, the term “probes” refers to a nucleic acid molecule which typically ranges in size from about 8 nucleotides to several hundred nucleotides in length. Such a molecule is typically used to identify a target nucleic acid sequence in a sample by hybridizing to such target nucleic acid sequence under stringent hybridization conditions. Generally, an oligonucleotide useful as a probe or primer that selectively hybridizes to a selected nucleotide sequence is at least about 15 nucleotides in length, usually at least about 18 nucleotides, and particularly about 21 nucleotides in length or more in length. Primer and probe design software programs are also commercially available, including without limitation, Primer Detective (ClonTech, Palo Alto, Calif.), Lasergene, (DNASTAR, Inc., Madison, Wis.); and Oligo software (National Biosciences, Inc., Plymouth, Minn.) and iOligo (Caesar Software, Portsmouth, N.H).

As used herein, a “sequence detection system” is any computational method in the art that can be used to analyze the results of a PCR reaction. One example is the Applied Biosystems HT7900 fast Real-Time PCR system. In certain embodiments, gene expression can be analyzed using, e.g., direct DNA expression in microarray, Sanger sequencing analysis, Northern blot, the Nanostring® technology, serial analysis of gene expression (SAGE), RNA-seq, tissue microarray, or protein expression with immunohistochemistry or western blot technique.

In an embodiment, the methods disclosed here further comprise determining a treatment regimen for the human subject. The methods could be used to generate a prescription treatment to treat, delay development or prevent progression of PD in an individual identified by the methods disclose herein as having PD. Furthermore, the method disclosed herein to adapt the correct or most appropriate treatment regimen and/or monitor the patient response to therapy.

In another aspect, the disclosure provides a method of treating a human subject for Parkinson's Disease (PD), the method comprising: (a) obtaining a diagnosis identifying a human subject as having PD, wherein the diagnosis was obtained by: (i) obtaining a blood sample from a human subject suspected of having PD; (ii) determining the expression level of at least one gene in the blood sample from the human subject suspected of having PD, wherein the at least one gene is selected from: SOD2, MT-ND1, TNF, IFNG, TP53, IL6, AKT1, HNF4A, HMOX1, FAS, APP, CYP17A1, IGF1, PTGS2, SOD1, BDNF, NOS2, TGM2, GCH1, UCHL1, IL1B, HNF1A, APOE, IGF2, CYP1A1, PPARG, SLC18A2, CD14, PINK1, INS, PARP1, NFKB1, SLC2A4, IDE, DRD2, GAD2, SORBS1, CP, TH, TSC2, PON1, E2F1, CXCR4, CDKN2A, KCNJ2, PPARGC1A, HGF, OPRM1, TF, ACE, CADM1, NQ01, GAD1, GH1, HFE, CXCL12, ABCB1, MAOB, BTG1, ABCC8, PDX1, ADH1C, CCL5, TCF7L2, ATF6, GPX1, CCL2, VDR, MTHFR, IL8, KIF11, MMP16, GSTM1, HP, NPPB, SEMA6A, NCAM2, SERPINB1, NAT2, MBNL1, PCDH18, OLFM4, RBMS3 and TBC1D22A; and (iii) comparing the expression level of the at least one gene expressed in the blood sample to the expression level of the at least one gene expressed in a non-PD, healthy control sample, whereby the increased or decreased expression level of the at least one gene expressed in the blood sample from the human subject suspected of having PD as compared to the non-PD sample is indicative of PD, thereby diagnosing the human subject as having PD; and (b) administering to the subject a PD treatment regimen.

In yet another aspect, the disclosure provides, a Parkinson's Disease diagnosis, prognosis or monitoring kit, consisting of a set of probes suitable for the detection and quantification of the nucleic acid expression of at least one gene selected from: SOD2, MTND1, TNF, IFNG, TP53, IL6, AKT1, HNF4A, HMOX1, FAS, APP, CYP17A1, IGF1, PTGS2, SOD1, BDNF, NOS2, TGM2, GCH1, UCHL1, IL1B, HNF1A, APOE, IGF2, CYP1A1, PPARG, SLC18A2, CD14, PINK1, INS, PARP1, NFKB1, SLC2A4, IDE, DRD2, GAD2, SORBS1, CP, TH, TSC2, PON1, E2F1, CXCR4, CDKN2A, KCNJ2, PPARGC1A, HGF, OPRM1, TF, ACE, CADM1, NQO1, GAD1, GH1, HFE, CXCL12, ABCB1, MAOB, BTG1, ABCC8, PDX1, ADH1C, CCL5, TCF7L2, ATF6, GPX1, CCL2, VDR, MTHFR, IL8, KIF11, MMP16, GSTM1, HP, NPPB, SEMA6A, NCAM2, SERPINB1, NAT2, MBNL1, PCDH18, OLFM4, RBMS3 and TBC1D22A.

In an embodiment, the present disclosure also relates to kits containing nucleic acid primers and kits containing nucleic acid primers and nucleic acid probes to diagnose and prognose PD in a sample of human thought to be afflicted with PD or known to be afflicted with PD. Such kit generally comprises a first container means having at least one oligonucleotide probe and/or primer that hybridizes to a target nucleic acid (e.g., SOD2 RNA) and a second container means containing at least one other oligonucleotide primer and/or probe that hybridizes to the above-mentioned nucleic acid specific sequences. The kit may further include other containers comprising additional components such as an additional oligonucleotide or primer and/or one or more of the following: buffers, reagents to be used in the assay (e.g., wash reagents, polymerases, internal controls or else) and reagents capable of detecting the presence of bound nucleic acid probe(s)/primer(s). Numerous embodiments of the kits of the present invention are possible. For example, the different container means can be divided in amplifying reagents and detection reagents. In addition, the kits may further include instructions for practicing the diagnostic and/or prognostic methods of the present disclosure. Such instructions can concern details relating to the experimental protocol as well as to the cut-off values for the PD specific biomarker ratio that may be used.

### EXAMPLES

### Methods

### Database Mining and Network Analysis

The DisGeNET database that integrates information from four respositories was queried: Online Medelian Inheritance in Man (OMIM), UniProt/SwissProte (UNIPROT), Pharmacogenomics Knowledge Base (PHARMGKB), and Comparative Toxicogenomics Database (CTD). DisGeNET can be accessed through the Cytoscape 2.8.3, a platform for complex network analysis. Search disease terms used in DisGeNET were the following: Parkinson Disease (umls:C0030567), Diabetes Mellitus, Type 2 (umls:C0011860). Disease-gene networks were retrieved for PD and T2DM independently. Using the advanced network merge option in Cytoscape, both PD and T2DM gene networks were merged using gene ID as a matching attribute. Only shared genes between both diseases were collected for further analysis.

The Disease and Gene Annotations database (DGA) was accessed through the world-wide-web (//dga.nubic.northwestern.edu/pages/search.php). Gene annotations shared between PD and T2DM were searched. Search disease terms in DGA were the following: Parkinson's disease, type 2 diabetes mellitus. The Human Experimental/Functional Mapper (HEFalMp) using the web-interface (//hefalmp.princeton.edu/) was used to investigate genetic associations between PD and T2DM. Search disease terms used in HEFalMp were: Parkinson disease, Diabetes Mellitus. A significance score of 10⁻⁵ wasused as a cut-off value for inclusion in the list of candidate genes. The Integrated Complex Traits Networks interface (iCTNet), can be accessed through the Cytoscape plugin. This database allows the automated construction of disease networks and integrates phenotype-SNP, protein-protein interaction, disease-tissue, tissue-gene and drug-gene interactions. Search disease terms were: Parkinson's disease, Diabetes Mellitus. The disease-gene networks associated with PD and T2DM were queried using a cutoff p-value of 10⁻⁵. Unlike DisGeNET, disease-gene networks are merged automatically in iCTNet. Like in previous steps, only shared genes between PD and T2DM were collected for further analysis. A total of 84 genes shared between PD and T2DM were collected from the aforementioned databases. Genetic associations were manually curated after searching the literature in Pubmed. Functional and gene ontology analysis was performed using GENEMANIA plugin in Cytoscape. In GENEMANIA, the default settings of 20 were used, which are the genes that have the greatest number of interactions, and advanced settings to include physical, predicted, and genetic interactions, and interconnected pathways.

### Gene Prioritization Methods and Cross-Validation Analysis

The list of 84 genes shared between PD and T2DM collected from the databases was used for subsequent analysis using GPEC, a Cytoscape 2.8.3 plugin that performs a RWR algorithm. The default, weighted and undirected human FLN was used for this analysis that contains 14,230 nodes and 263,884 links. Nodes represent genes and each link represents the likelihood that the connected genes participate in common biological processes. In order to perform the gene prioritization in GPEC, a list of well-characterized genes associated with PD and T2DM was first collected and genes involved in the PD and T2DM KEGG pathways (Table 1). Well-characterized genes known to be associated with PD and T2DM were retrieved from the OMIM (//www.ncbi.nlm.nih.gov/omim), the Genetic association database (GAD) (//geneticassociationdb.nih.gov/) and PDgene (//www.pdgene.org/) (Table 1). Genes involved in the PD and T2DM signaling pathways were retrieved from the KEGG database (//www.genome.jp/kegg/pathway.html).

TABLE 1
Curated gene sets used for RWR prioritization.

| Disease or | |
|---|---|
| biological | |
| pathway | Gene sets |
| Parkinson's | KEGG 05012: PD signaling pathway |
| disease | PDgene, GAD, OMIM: GAK, DGKQ, STH, MAPT, |
| | LRRK2, SNCA, LOC642072, WNT3, RIT2, GBA, |
| | MCCC1, LAMP3, SCARB2, SYT11, ACMSD, |
| | STK39, BST1, HLA-DRB5, CCDC62, HIP1R, |
| | HLA-DRA, PARK16, SLC45A3, NUCKS1, |
| | RAB7L1, SLC41A1, PM20D1, C17ORF69, |
| | KIAA1267,LOC644246, NSF, FAM47E, SREBF1, |
| | TMEM175, BRDG1, DLG2, PLEKHM1, IMP5, |
| | CRHR1, PM20D1 |
| Type 2 diabetes | KEGG 04930: T2DM signaling pathway |
| | GAD and OMIM: ARF5, PAX4, SND1, IGF2BP2, |
| | GRK5, RASGRP1, GLIS3, CDKN2B, CDC123, |
| | HNF1B, FAM58A, DUSP9, CDKAL1, LAMA1, |
| | FTO, HHEX, RBM43, RND3, MAEA, GLIS3, |
| | FITM2, R3HDML, GCC1, PSMD6, ZFAND3, |
| | HMG20A, AP3S2, KCNQ1, SPRY2, C2CD4A, |
| | C2CD4B, BCL11A, ZBED3, KLF14, TP53INP1, |
| | CENTD2, HMGA2, ZFAND6, PRC1, IRS1, |
| | MTNR1B, JAZF1, IDE, SRR, PTPRD, SLC30A8, |
| | CAMK1D, TSPAN8, LGR5, THADA |
| Insulin | KEGG: 04910 |
| signaling | |
| Nitric oxide | MSigDB: M11650 BioCarta: Nitric oxide signaling |
| biosynthesis | pathway |
| Glucose | MSigDB: M1879 Reactome glucose metabolism |
| metabolism | |
| Inflammation | KEGG: 04062 |
| Lipid | KEGG: 00071 |
| metabolism | |

As a first step in the prioritization, the list of well-characterized genes associated with PD and the PD KEGG pathway was used as a training set. The test set included the 84 genes shared between both diseases and genes associated with T2DM and its associated KEGG pathway. The training set was manually curated to ensure that there was no overlap with any of the genes contained in the test set. To perform the RWR, back-probability was set to 0.5 and candidate genes were scored and ranked. As a second step, a series of prioritization steps was performed with respect to the most significant biological pathways retrieved by GENEMANIA. These prioritization steps were performed for each individual pathway independently. To this end, the set of genes curated was collected for each biological pathway from the Broad Institute's Molecular Signatures Database (MSigDB) 3.0 (Table 1). Here, the training set consisted of genes curated for each pathway and the test set consisted of the 84 genes shared between PD and T2DM. In GPEC, the performance of each prioritization was evaluated with a leave-one-out cross-validation (LOOCV) strategy where the number of training genes is equal to the number of cross-validation trials and one of the genes in the test set is held out during each trial. As a result, a ROC curve of sensitivity versus 1-specificity is built by the software. Since all the scores were determined by the RWR algorithm, the final score for each gene was defined as the sum of all individual scores obtained from each prioritization as previously demonstrated using similar analyses. The overall workflow is presented in FIG. 1.

### Information About HBS and PROBE Study Participants

The Institutional Review Boards of Rosalind Franklin University of Medicine and Science approved the study protocol. Written informed consent was received from all participants. 96 individuals were enrolled, including 50 PD patients (31 men, 19 women; Hoehn and Yahr scale 1.97±0.62; mean age at enrollment 63.12±8.96; mean age at onset 58.75±10.17) and 46 healthy age-matched controls (HC) (26 men, 20 women; mean age at enrollment 64.28±10.42) enrolled in the Harvard Biomarker Study (HBS). Other clinical information is reported in Santiago & Potashkin JA (2013) Integrative network analysis unveils convergent molecular pathways in Parkinson's disease and diabetes. PloS one 8: e83940. There were 5 PD and 5 HC patients with T2DM. Details of patient and controls recruitment, clinical assessments, and biobanking in the HBS study population have been reported in part elsewhere Ding et al., (2011) Association of SNCA with Parkinson: replication in the Harvard NeuroDiscovery Center Biomarker Study. Movement disorders: Official Journal of the Movement Disorder Society 26: 2283-2286. As an independent replication set, 51 PD patients were used (29 men, 22 women; mean age at enrollment 63.16±6.42; Hoehn and Yahr scale 2±0.28) and 45 HC (24 male, 21 women; mean age at enrollment 65.12±8.60) enrolled in the PROBE Study (#NCT00653783). There was one HC patient with T2DM. Clinical diagnosis of PD was based on the United Kingdom Parkinson's Disease Society Brain Bank criteria. Healthy controls had no history of neurological disease and a Mini-Mental State Examination (MMSE) test score higher than 27. Inclusion and exclusion criteria for patients enrolled in the PROBE study are reported elsewhere in Potashkin et al., (2012) Biosignatures for Parkinson's disease and atypical parkinsonian disorders patients. PloS one 7: e43595.

### RNA Isolation and Real Time Polymerase Chain Reactions

Blood was collected and prepared as described using the PAXgene Blood RNA system (Qiagen,Valencia, Calif., USA) (see Scherzer et al. (2007) Molecular markers of early Parkinson's disease based on gene expression in blood. Proceedings of the National Academy of Sciences of the United States of America 104: 955-960). Samples with RNA integrity values >7.0 and ratio of absorbances at 260/280 nm between 1.7 and 2.4 were used in the current study. Primer Express software (Life Technologies, Carlsbad, Calif., USA) was used to design the primers. The High Capacity RNA transcription kit (Life Technologies, Carlsbad, Calif., USA) was used to reverse transcribe 1 μg of total RNA according to the manufacturer's protocol. The DNA engine Opticon 2 Analyzer (Bio-Rad Life Sciences, Hercules, Calif., USA) was used for the qPCR reactions. Each 25 μl reaction contained Power SYBR (Life Technologies, Carlsbad, Calif., USA) and primers at a concentration of 5 μM. Primer sequences used in qPCR assays are as follows: GAPDH; forward: 5′-CAACGGATTTGGTCGTATTGG-3′ (SEQ ID NO: 03); reverse: 5′-TGATGGCAACAATATCCACTTTACC-3′(SEQ ID NO: 04), SOD2; forward: 5′-GTTCAATGGTGGTGGTCATATCA-3′(SEQ ID NO: 05); reverse: 5′-GCAACTCCCCTTTGGGTTCT-3′(SEQ ID NO: 06). Amplification conditions and detailed description of qPCR experiments is described in Santiago & Potashkin (2013) Integrative network analysis unveils convergent molecular pathways in Parkinson's disease and diabetes. PloS one 8: e83940.

### Statistical Analysis

All analyses were performed with Prism4.0 (GraphPad, La Jolla, Calif., USA) and Statistica 8.0 (Statsoft, Okla., Tulsa, USA). A student t-test (two-tailed) was used to estimate the significance between PD cases and controls for numerical variables. Linear regression and Pearson correlation analysis was used to determine statistical significance for the prospective biomarker adjusting for sex, age, Hoehn & Yahr scale in both cohorts of patients and body mass index (BMI) in the HBS study. A ROC curve analysis was used to evaluate the diagnostic accuracy. A p-value less than 0.05 was regarded statistically significant.

### Example 1

### Identification of Shared Genes Between PD and T2DM from Disease-Gene Databases.

In DISGENET, the central node represents the disease and the nodes linked to the central node represent genes that have been associated to the queried disease. The disease-gene networks associated with both PD and T2DM were queried. Analysis of the merged network revealed a cluster consisting of 53 shared genes between PD and T2DM (Table 2).

TABLE 2
PD and T2DM shared cluster of genes.

| Gene Symbol | Entrez ID | Database |
|---|---|---|
| VDR | 7421 | DisGeNET, DGA |
| TNF | 7124 | DisGeNET, DGA |
| TH | 7054 | DisGeNET, DGA |
| TF | 7018 | DisGeNET, DGA |
| SOD2 | 6648 | DisGeNET, DGA |
| PTGS2 | 5743 | DisGeNET, DGA |
| PON1 | 5444 | DisGeNET, DGA |
| PINK1 | 65018 | DisGeNET, DGA |
| PARP1 | 142 | DisGeNET, DGA |
| NQO1 | 1728 | DisGeNET, DGA |
| MTND1 | 4535 | DisGeNET, DGA |
| NAT2 | 10 | DisGeNET, DGA |
| MTHFR | 4524 | DisGeNET, DGA |
| INS | 3630 | DisGeNET, DGA |
| IL1B | 3553 | DisGeNET, DGA |
| IFNG | 3458 | DisGeNET, DGA |
| IL1B | 3553 | DisGeNET, DGA |
| HP | 3240 | DisGeNET, DGA |
| HMOX1 | 3162 | DisGeNET, DGA |
| HFE | 3077 | DisGeNET, DGA |
| GPX1 | 2876 | DisGeNET, DGA |
| FAS | 355 | DisGeNET, DGA |
| DRD2 | 1813 | DisGeNET, DGA |
| CYP1A1 | 1543 | DisGeNET, DGA |
| CYP17A1 | 1586 | DisGeNET, DGA |
| CDKN2A | 1029 | DisGeNET, DGA |
| CD14 | 929 | DisGeNET, DGA |
| CCL5 | 6352 | DisGeNET, DGA |
| CCL2 | 6347 | DisGeNET, DGA |
| APOE | 348 | DisGeNET, DGA |
| ACE | 1636 | DisGeNET, DGA |
| ABCB1 | 5243 | DisGeNET, DGA |
| AKT1 | 207 | DisGeNET, DGA |
| CP | 1356 | DisGeNET, DGA |
| GCH1 | 2643 | DisGeNET, DGA |
| GSTM1 | 2944 | DisGeNET, DGA |
| IGF1 | 3479 | DisGeNET, DGA |
| IL8 | 3576 | DisGeNET, DGA |
| MAOB | 4129 | DisGeNET, DGA |
| SLC18A2 | 6571 | DisGeNET, DGA |
| SOD1 | 6647 | DisGeNET, DGA |
| UCHL1 | 7345 | DisGeNET, DGA |
| OPRM1 | 4988 | DisGeNET |
| NPPB | 4879 | DisGeNET |
| NOS2 | 4843 | DisGeNET |
| NFKB1 | 4790 | DisGeNET |
| IL6 | 3569 | DisGeNET |
| IDE | 3416 | DisGeNET |
| HGF | 3082 | DisGeNET |
| BDNF | 627 | DisGeNET |
| ATF6 | 22926 | DisGeNET |
| ADH1C | 126 | DisGeNET |
| APP | 351 | DGA |
| E2F1 | 1869 | DGA |
| GAD1 | 2571 | DGA |
| GAD2 | 2572 | DGA |
| GH1 | 2688 | DGA |
| TGM2 | 7052 | DGA |
| TP53 | 7157 | DGA |
| TSC2 | 7249 | DGA |
| BTG1 | 694 | iCTNet |
| HNF4A | 3172 | iCTNet, HEFalMp |
| SEMA6A | 57556 | iCTNet |
| CXCR4 | 7852 | iCTNet |
| RBMS3 | 27303 | iCTNet |
| PPARG | 5468 | iCTNet, DGA |
| HNF1A | 6927 | iCTNet |
| TCF7L2 | 6934 | iCTNet |
| TBC1D22A | 25771 | iCTNet, DisGeNET |
| OLFM4 | 10562 | iCTNet |
| SORBS1 | 10580 | iCTNet |
| CADM1 | 23705 | iCTNet |
| PCDH18 | 54510 | iCTNet |
| NCAM2 | 4685 | iCTNet |
| MMP16 | 4325 | iCTNet |
| SERPINB1 | 1992 | iCTNet |
| PPARGC1A | 10891 | iCTNet, DGA |
| MBNL1 | 4154 | iCTNet |
| KIF11 | 3832 | iCTNet |
| KCNJ2 | 3759 | iCTNet |
| CXCL12 | 6387 | iCTNet |
| PDX1 | 3651 | HEFalMp |
| SLC2A4 | 6517 | HEFalMp |
| ABCC8 | 6833 | HEFalMp |

The DGA interface was next explored (see Peng et al., (2013) The Disease and Gene Annotations (DGA): an annotation resource for human disease. Nucleic Acids Research 41: D553-560) and found 42 overlapping genes with the gene set collected in DisGeNET and 8 additional genes shared between PD and T2DM (Table 2). Next, the HEFalMp interface was interrogated (see Huttenhower et al., (2009) Exploring the human genome with functional maps. Genome Research 19: 1093-1106). Similarly to DGA and DisGeNET, the shared genes between PD and T2DM were collected. The most significant genes in T2DM associated to PD were HNF4A, PDX1, SLC2A4, and ABCC8 (Q<10^{-0.5}) (Table 2). Finally, the iCTNet interface was interrogated (see Wang et al., (2011) iCTNet: a Cytoscape plugin to produce and analyze integrative complex traits networks. BMC Bioinformatics 12: 380). The iCTNet contains results from 118 GWAS published studies and data from the GWAS catalog. In iCTNet, 20 genes shared between both diseases (Table 2) were found. A total of 84 genes shared between PD and T2DM were collected from the aforementioned databases and used for further analysis.

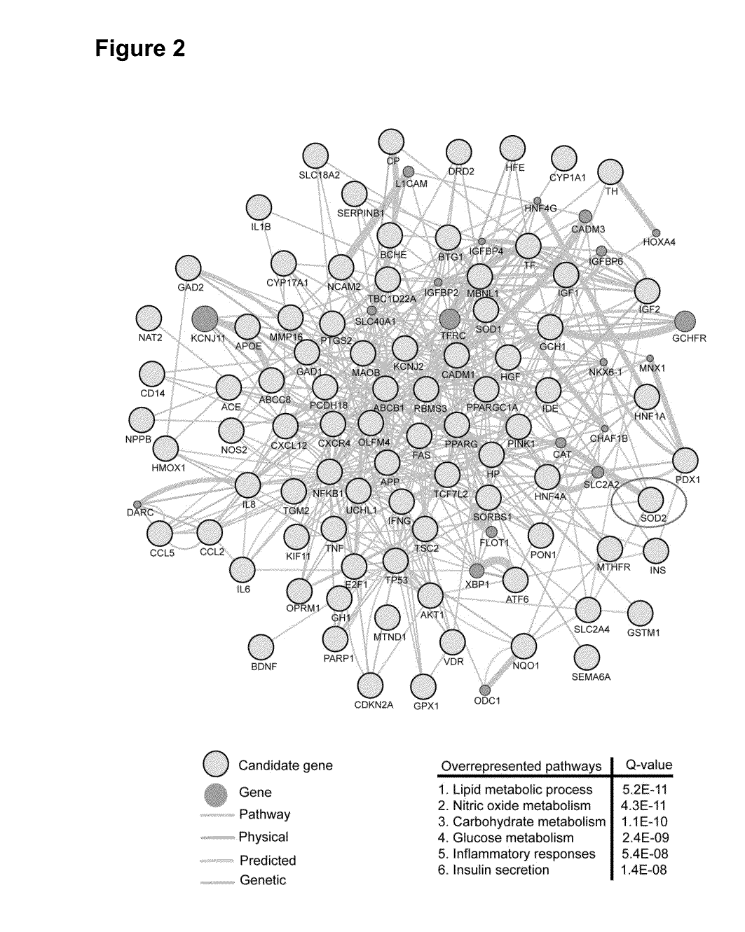

To further identify the potential functional implications in the cluster of genes shared between PD and T2DM, all 84 genes were imported into GeneMANIA (Montojo et al., (2010) GeneMANIA Cytoscape plugin: fast gene function predictions on the desktop. Bioinformatics 26: 2927-2928). Analysis of the 84 shared genes identified the most overrepresented pathways including nitric oxide biosynthetic processing, carbohydrate and lipid metabolic processing, insulin secretion, regulation of glucose, and inflammation (FIG. 2).

### Gene Prioritization and Experimental Validation

Given the numerous molecular links between PD and T2DM, the extent to which genes identified as shared between PD and T2DM can be used to classify patients with PD was investigated. This idea is particularly salient in light of the recent finding that revealed that genes identified in shared molecular networks between PD and T2DM may improve the clinical diagnosis of PD. Accordingly, APP was identified in a functional network shared between well-characterized genes associated with PD and T2DM. APP mRNA was capable to distinguish PD patients from HC with 80% accuracy (see Santiago & Potashkin (2013) Integrative network analysis unveils convergent molecular pathways in Parkinson's disease and diabetes. PLoS One 8: e83940), a diagnostic capacity that extends beyond the one afforded by the current clinical diagnostic criteria.

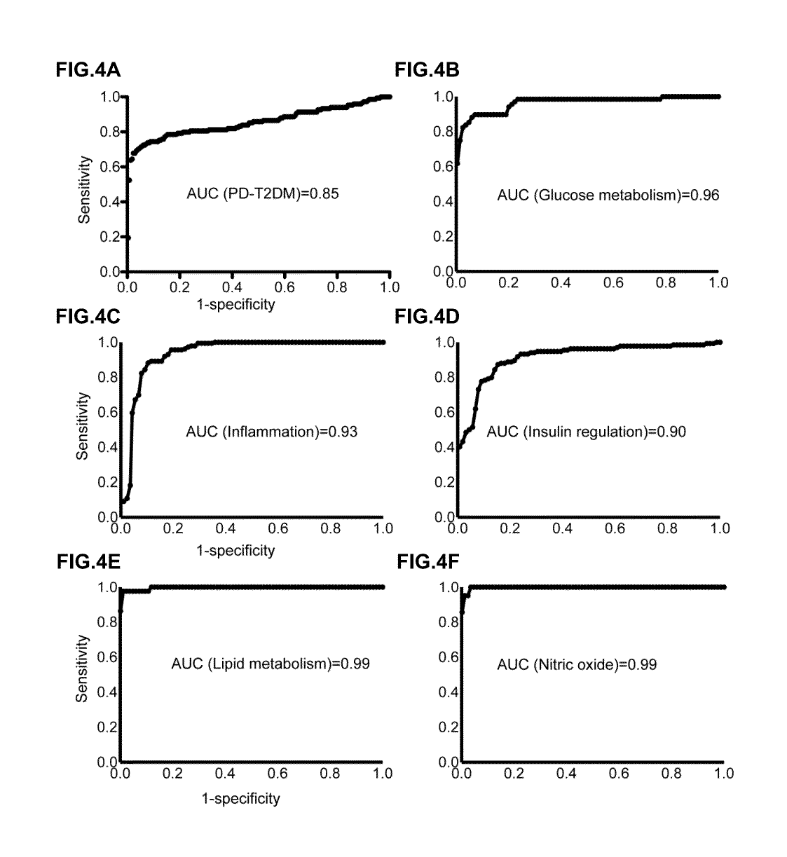

A candidate prioritization approach was implemented using a RWR algorithm within the human the FLN described previously (Santiago & Potashkin (2013) Integrative network analysis unveils convergent molecular pathways in Parkinson's disease and diabetes. PLoS One 8: e83940; Santiago & Potashkin (2014) A network approach to diagnostic biomarkers in progressive supranuclear palsy. Movement Disorders: official journal of the Movement Disorder Society 29: 550-555; Gao et al. (2014) Discovery of the neuroprotective effects of alvespimycin by computational prioritization of potential anti-parkinson agents. The FEBS Journal 281(4):1110-22 (Epub Jan. 9, 2014); and Kohler et al., (2008) Walking the interactome for prioritization of candidate disease genes. American Journal of Human Genetics 82: 949-958). This algorithm measures the closeness of potentially candidate genes to confirmed genes within the FLN or protein-protein interaction network. GPEC, a cytoscape plugin for RWR-based gene prioritization was used (Le & Kwon (2012) GPEC: a Cytoscape plug-in for random walk-based gene prioritization and biomedical evidence collection. Computational Biology and Chemistry 37: 17-23) to rank 84 candidates collected from the curated databases (Table 2). In the RWR algorithm, the known disease genes are mapped to the FLN and specified as “training set” and the “test set” containing potential candidates can be ranked according to their closeness to the training genes within the FLN (See Methods). The training set consisted of well-characterized genes associated with PD and its KEGG associated pathway. The test set included the list of 84 shared genes and well-characterized genes associated with T2DM and its KEGG associated pathway. RWR score-based genes are listed in Table 3. Further, the performance of the gene prioritization using a LOOCV strategy (see Methods) was evaluated. LOOCV represented in terms of receiver operating characteristic curve (ROC) resulted in an area under curve AUC_{PD-T2DM} value of 0.85 (FIG. 4A).

TABLE 3
RWR-based scores for each prioritization within the functional linkage network.

| | | Score | | | | | | |
|---|---|---|---|---|---|---|---|---|
| Rank | Gene | (PD-T2DM) | Score (p1) | Score (p2) | Score (p3) | Score (p4) | Score (p5) | Score (c) |
| 1 | SOD2 | 9.07E−04 | 1.49E−04 | 2.21E−04 | 1.37E−03 | 3.73E−05 | 3.96E−04 | 3.08E−03 |
| 2 | MTND1 | 2.68E−03 | 1.41E−05 | 1.16E−05 | 1.47E−04 | 1.94E−06 | 7.71E−05 | 2.93E−03 |
| 3 | TNF | 2.18E−04 | 4.75E−04 | 1.38E−04 | 2.46E−04 | 1.76E−03 | 5.96E−05 | 2.90E−03 |
| 4 | IFNG | 7.27E−05 | 2.99E−04 | 7.12E−05 | 5.00E−05 | 1.88E−03 | 2.39E−05 | 2.39E−03 |
| 5 | TP53 | 5.18E−04 | 5.58E−04 | 2.70E−04 | 3.90E−04 | 3.96E−04 | 2.26E−04 | 2.36E−03 |
| 6 | IL6 | 7.38E−05 | 3.67E−04 | 1.01E−04 | 5.93E−05 | 1.53E−03 | 3.17E−05 | 2.16E−03 |
| 7 | AKT1 | 2.10E−04 | 1.20E−03 | 5.16E−04 | 0.00E+00 | 0.00E+00 | 3.20E−05 | 1.96E−03 |
| 8 | HNF4A | 4.02E−05 | 6.02E−04 | 2.89E−05 | 7.33E−04 | 2.42E−05 | 3.74E−04 | 1.80E−03 |
| 9 | HMOX1 | 1.47E−04 | 5.53E−05 | 3.04E−04 | 4.63E−05 | 3.48E−05 | 1.18E−03 | 1.77E−03 |
| 10 | FAS | 1.87E−04 | 2.52E−04 | 6.25E−04 | 1.70E−04 | 2.69E−04 | 3.20E−05 | 1.53E−03 |
| 11 | APP | 3.17E−04 | 4.73E−04 | 1.39E−04 | 1.17E−04 | 1.97E−04 | 9.77E−05 | 1.34E−03 |
| 12 | CYP17A1 | 2.99E−05 | 4.50E−05 | 2.06E−04 | 2.97E−05 | 4.16E−05 | 8.76E−04 | 1.23E−03 |
| 13 | IGF1 | 3.50E−05 | 4.96E−04 | 6.35E−05 | 5.54E−05 | 3.60E−04 | 1.53E−05 | 1.03E−03 |
| 14 | PTGS2 | 1.40E−04 | 2.16E−04 | 2.90E−04 | 2.70E−05 | 2.05E−04 | 1.39E−04 | 1.02E−03 |
| 15 | SOD1 | 2.89E−04 | 2.55E−05 | 2.22E−04 | 2.10E−04 | 2.70E−05 | 2.07E−04 | 9.80E−04 |
| 16 | BDNF | 1.97E−05 | 5.17E−05 | 6.01E−04 | 2.24E−05 | 1.01E−04 | 5.04E−05 | 8.46E−04 |
| 17 | NOS2 | 3.27E−05 | 5.20E−05 | 4.59E−04 | 2.52E−05 | 1.45E−04 | 1.21E−04 | 8.34E−04 |
| 18 | TGM2 | 2.12E−05 | 9.00E−05 | 3.31E−05 | 7.09E−06 | 3.18E−05 | 5.03E−04 | 6.86E−04 |
| 19 | GCH1 | 1.48E−04 | 4.13E−05 | 9.22E−06 | 2.58E−04 | 4.14E−06 | 2.06E−04 | 6.66E−04 |
| 20 | UCHL1 | 1.29E−04 | 7.90E−07 | 6.00E−07 | 5.28E−04 | 9.60E−07 | 1.30E−07 | 6.60E−04 |
| 21 | IL1B | 2.90E−05 | 5.13E−05 | 2.85E−05 | 2.10E−05 | 5.10E−04 | 7.69E−06 | 6.48E−04 |
| 22 | HNF1A | 1.88E−05 | 1.35E−04 | 1.69E−05 | 2.00E−04 | 3.02E−05 | 1.81E−04 | 5.82E−04 |
| 23 | APOE | 5.10E−05 | 1.28E−04 | 6.59E−05 | 4.33E−05 | 2.45E−04 | 1.91E−05 | 5.52E−04 |
| 24 | IGF2 | 2.88E−05 | 2.32E−04 | 2.61E−05 | 1.65E−04 | 6.39E−05 | 1.35E−05 | 5.29E−04 |
| 25 | CYP1A1 | 1.42E−05 | 1.04E−05 | 3.93E−05 | 1.18E−05 | 5.55E−06 | 4.47E−04 | 5.28E−04 |
| 26 | PPARG | 7.15E−05 | 2.07E−04 | 1.79E−05 | 1.30E−04 | 7.45E−05 | 1.56E−05 | 5.16E−04 |
| 27 | SLC18A2 | 4.16E−04 | 4.01E−05 | 2.72E−05 | 8.78E−06 | 5.75E−06 | 6.66E−06 | 5.05E−04 |
| 28 | CD14 | 2.29E−04 | 3.94E−05 | 1.91E−05 | 1.14E−05 | 1.96E−04 | 6.13E−06 | 5.01E−04 |
| 29 | PINK1 | 9.85E−05 | 1.25E−04 | 8.76E−05 | 5.50E−05 | 8.71E−05 | 2.01E−06 | 4.55E−04 |
| 30 | INS | 8.21E−05 | 0.00E+00 | 7.97E−05 | 1.30E−04 | 1.00E−04 | 1.88E−05 | 4.10E−04 |
| 31 | PARP1 | 1.81E−04 | 7.52E−05 | 3.56E−05 | 2.93E−05 | 5.59E−05 | 1.60E−05 | 3.93E−04 |
| 32 | NFKB1 | 1.62E−04 | 1.34E−04 | 5.60E−05 | 3.34E−05 | 0.00E+00 | 6.88E−06 | 3.92E−04 |
| 33 | SLC2A4 | 1.88E−04 | 0.00E+00 | 1.59E−05 | 6.06E−05 | 1.55E−05 | 9.41E−05 | 3.74E−04 |
| 34 | IDE | 9.91E−05 | 7.13E−05 | 1.21E−05 | 5.38E−05 | 1.49E−05 | 1.21E−04 | 3.72E−04 |
| 36 | DRD2 | 1.09E−04 | 4.91E−05 | 6.36E−05 | 1.56E−05 | 1.20E−04 | 8.04E−06 | 3.66E−04 |
| 37 | GAD2 | 1.79E−05 | 7.99E−06 | 1.68E−05 | 2.67E−04 | 1.21E−05 | 2.78E−05 | 3.50E−04 |
| 38 | SORBS1 | 3.21E−05 | 0.00E+00 | 1.04E−04 | 1.30E−05 | 1.96E−04 | 4.86E−06 | 3.50E−04 |
| 39 | CP | 1.43E−04 | 1.61E−05 | 2.32E−05 | 2.32E−05 | 1.19E−05 | 1.18E−04 | 3.35E−04 |
| 40 | TH | 1.64E−04 | 5.27E−06 | 3.49E−06 | 1.32E−04 | 2.75E−06 | 8.15E−06 | 3.16E−04 |
| 41 | TSC2 | 1.53E−05 | 0.00E+00 | 3.85E−05 | 6.54E−05 | 1.80E−04 | 3.69E−06 | 3.02E−04 |
| 42 | PON1 | 7.71E−06 | 8.29E−05 | 1.48E−05 | 1.73E−04 | 5.60E−06 | 7.00E−06 | 2.91E−04 |
| 35 | E2F1 | 3.08E−05 | 1.17E−04 | 1.95E−05 | 3.01E−05 | 8.21E−05 | 5.79E−06 | 2.85E−04 |
| 43 | CXCR4 | 3.42E−05 | 1.74E−04 | 4.56E−05 | 1.89E−05 | 0.00E+00 | 5.69E−06 | 2.78E−04 |
| 44 | CDKN2A | 4.95E−05 | 8.19E−05 | 3.34E−05 | 2.77E−05 | 5.81E−05 | 7.88E−06 | 2.59E−04 |
| 45 | KCNJ2 | 3.68E−05 | 9.63E−06 | 1.80E−04 | 4.50E−06 | 7.34E−06 | 8.90E−07 | 2.39E−04 |
| 46 | PPARGC1A | 8.59E−05 | 0.00E+00 | 1.60E−05 | 9.91E−05 | 1.93E−05 | 1.05E−05 | 2.31E−04 |
| 47 | HGF | 1.67E−05 | 4.80E−05 | 2.10E−05 | 1.04E−05 | 8.99E−05 | 8.56E−06 | 1.95E−04 |
| 48 | OPRM1 | 6.93E−05 | 1.64E−05 | 3.46E−05 | 3.93E−06 | 6.48E−05 | 5.90E−07 | 1.90E−04 |
| 49 | TF | 4.58E−05 | 3.04E−05 | 2.36E−05 | 2.87E−05 | 2.40E−05 | 2.41E−05 | 1.77E−04 |
| 50 | ACE | 2.33E−05 | 2.06E−05 | 2.16E−05 | 3.08E−05 | 6.03E−05 | 1.68E−05 | 1.73E−04 |
| 51 | CADM1 | 8.86E−06 | 4.13E−05 | 8.41E−05 | 4.55E−06 | 3.34E−05 | 1.18E−06 | 1.73E−04 |
| 52 | NQO1 | 2.52E−05 | 4.81E−06 | 8.78E−06 | 5.38E−05 | 1.76E−06 | 3.06E−05 | 1.25E−04 |
| 53 | GAD1 | 1.45E−05 | 5.59E−06 | 1.94E−05 | 4.08E−05 | 1.15E−06 | 4.07E−05 | 1.22E−04 |
| 54 | GH1 | 4.26E−06 | 6.78E−05 | 6.50E−06 | 1.13E−05 | 2.16E−05 | 3.76E−06 | 1.15E−04 |
| 55 | HFE | 6.71E−05 | 7.37E−06 | 6.62E−06 | 1.02E−05 | 8.94E−06 | 1.20E−05 | 1.12E−04 |
| 56 | CXCL12 | 1.68E−05 | 4.53E−05 | 2.75E−05 | 1.38E−05 | 0.00E+00 | 5.87E−06 | 1.09E−04 |
| 57 | ABCB1 | 2.46E−05 | 1.12E−05 | 8.73E−06 | 9.51E−06 | 4.93E−06 | 3.38E−05 | 9.27E−05 |
| 58 | MAOB | 2.62E−05 | 4.99E−06 | 6.56E−06 | 1.26E−05 | 2.86E−06 | 3.83E−05 | 9.15E−05 |
| 59 | BTG1 | 4.42E−06 | 2.90E−05 | 8.75E−06 | 3.89E−06 | 3.93E−05 | 1.25E−06 | 8.66E−05 |
| 60 | ABCC8 | 4.46E−06 | 2.51E−05 | 3.57E−05 | 9.80E−06 | 2.23E−06 | 8.63E−06 | 8.58E−05 |
| 61 | PDX1 | 6.06E−06 | 3.74E−05 | 8.36E−06 | 1.61E−05 | 9.14E−06 | 4.64E−06 | 8.16E−05 |
| 62 | ADH1C | 7.61E−06 | 3.92E−05 | 7.80E−07 | 3.25E−05 | 3.70E−07 | 0.00E+00 | 8.04E−05 |
| 63 | CCL5 | 1.44E−05 | 3.18E−05 | 1.91E−05 | 8.35E−06 | 0.00E+00 | 2.33E−06 | 7.60E−05 |
| 64 | TCF7L2 | 1.13E−05 | 2.25E−05 | 9.84E−06 | 8.55E−06 | 1.77E−05 | 3.25E−06 | 7.32E−05 |
| 65 | ATF6 | 1.62E−05 | 1.71E−05 | 1.00E−05 | 6.65E−06 | 1.39E−05 | 2.80E−06 | 6.66E−05 |
| 66 | GPX1 | 3.84E−05 | 2.84E−06 | 2.18E−06 | 1.00E−05 | 1.50E−06 | 1.06E−05 | 6.56E−05 |
| 67 | CCL2 | 1.04E−05 | 2.89E−05 | 1.42E−05 | 7.16E−06 | 0.00E+00 | 2.60E−06 | 6.32E−05 |
| 68 | VDR | 8.29E−06 | 1.41E−05 | 6.71E−06 | 8.37E−06 | 8.68E−06 | 1.56E−05 | 6.18E−05 |
| 69 | MTHFR | 1.12E−05 | 4.88E−06 | 1.88E−06 | 2.98E−05 | 2.32E−06 | 1.12E−05 | 6.12E−05 |
| 70 | IL8 | 1.01E−05 | 2.55E−05 | 1.21E−05 | 1.00E−05 | 0.00E+00 | 2.76E−06 | 6.04E−05 |
| 71 | KIF11 | 1.11E−05 | 1.34E−05 | 1.79E−05 | 7.16E−06 | 9.12E−06 | 1.18E−06 | 5.99E−05 |
| 72 | MMP16 | 2.99E−06 | 4.76E−06 | 3.01E−06 | 3.24E−06 | 1.65E−05 | 1.51E−06 | 3.20E−05 |
| 73 | GSTM1 | 1.11E−05 | 1.73E−06 | 1.33E−06 | 3.94E−06 | 5.70E−07 | 1.29E−05 | 3.16E−05 |
| 74 | HP | 5.81E−06 | 4.87E−06 | 3.74E−06 | 3.76E−06 | 8.82E−06 | 1.60E−06 | 2.86E−05 |
| 75 | NPPB | 8.80E−07 | 2.50E−06 | 6.81E−06 | 5.40E−07 | 1.64E−05 | 4.70E−07 | 2.76E−05 |
| 76 | SEMA6A | 5.00E−07 | 3.89E−06 | 2.06E−06 | 3.60E−07 | 4.98E−06 | 9.00E−08 | 1.19E−05 |
| 77 | NCAM2 | 1.28E−06 | 2.10E−06 | 2.95E−06 | 3.50E−07 | 3.00E−06 | 1.30E−07 | 9.81E−06 |
| 78 | SERPINB1 | 2.33E−06 | 1.53E−06 | 6.10E−07 | 1.59E−06 | 2.97E−06 | 5.20E−07 | 9.55E−06 |
| 79 | NAT2 | 1.29E−06 | 2.90E−07 | 1.20E−07 | 2.30E−06 | 4.00E−08 | 1.46E−06 | 5.50E−06 |
| 80 | MBNL1 | 4.50E−07 | 1.56E−06 | 8.20E−07 | 7.00E−07 | 6.30E−07 | 1.10E−07 | 4.27E−06 |
| 81 | PCDH18 | 1.80E−07 | 1.51E−06 | 8.60E−07 | 1.10E−07 | 1.46E−06 | 4.00E−08 | 4.16E−06 |
| 82 | OLFM4 | 8.00E−08 | 0.00E+00 | 1.80E−06 | 8.00E−08 | 8.00E−08 | 1.00E−08 | 2.05E−06 |
| 83 | RBMS3 | 1.20E−07 | 2.50E−07 | 1.10E−07 | 1.37E−06 | 6.00E−08 | 6.00E−08 | 1.97E−06 |
| 84 | TBC1D22A | 2.30E−07 | 1.40E−07 | 4.10E−07 | 2.30E−07 | 5.00E−08 | 6.00E−08 | 1.12E−06 |

RWR-based scores for each prioritization within the functional linkage network. Score PD-T2DM is the score for the disease prioritization, p1 is insulin signaling pathway, p2 is nitric oxide biosynthesis, p3 is glucose metabolism, p4 is inflammation, p5 is lipid metabolism and c is the cumulative score.

As a second step, the list of 84 shared genes was prioritized with respect to the most significant biological pathways determined by GeneMania (see Methods). The set of genes curated for each biological pathway was collected from the Broad Institute's Molecular Signatures Database 3.0 (MSigDB) (see Subramanian et al., (2005) Gene set enrichment analysis: a knowledge-based approach for interpreting genome-wide expression profiles. Proceedings of the National Academy of Sciences of the United States of America 102: 15545-15550) (Table 1). These gene sets were used as training sets during each prioritization. Gene prioritization was performed in GPEC for each individual pathway independently (Methods, Table 3). LOOCV performed for each prioritization resulted in AUC values ranging from 0.90-0.99 (FIG. 4B-E). The top RWR score-based genes are listed in Table 4. The complete list of RWR score-based candidate genes according to each prioritization step is provided in (Table 3).

TABLE 4
Highly ranked RWR score-based genes.

| | Gene | | |
|---|---|---|---|
| Rank | Symbol | Gene Name | Score |
| 1 | SOD2 | Superoxide dismutase 2 | 3.08E−03 |
| 2 | MT-ND1 | Mitochondrially encoded NADH | 2.93E−03 |
| | | dehydrogenase 1 | |
| 3 | IFNG | Interferon, gamma | 2.90E−03 |
| 4 | TNF | Tumor necrosis factor | 2.39E−03 |
| 5 | TP53 | Tumor protein p53 | 2.36E−03 |
| 6 | IL6 | Interleukin 6 | 2.16E−03 |
| 7 | AKT1 | V-akt murine thymoma viral oncogene | 1.96E−03 |
| | | homolog 1 | |
| 8 | HNF4A | Hepatocyte nuclear factor 4, alpha | 1.80E−03 |
| 9 | HMOX1 | Heme oxygenase (decycling) 1 | 1.77E−03 |
| 10 | FAS | Fas (TNF receptor superfamily, | 1.53E−03 |
| | | member 6) | |
| 11 | APP | Amyloid beta (A4) precursor protein | 1.34E−03 |
| 12 | CYP17A1 | Cytochrome P450, family 17, | 1.23E−03 |
| | | subfamily A, polypeptide 1 | |
| 13 | IGF1 | Insulin-like growth factor 1 | 1.03E−03 |
| 14 | PTGS2 | Prostaglandin-endoperoxide synthase 2 | 1.02E−03 |
| 15 | SOD1 | Superoxide dismutase 1, soluble | 9.80E−04 |
| 16 | BDNF | Brain-derived neurotrophic factor | 8.46E−04 |
| 17 | NOS2 | Nitric oxide synthase 2 | 8.34E−04 |
| 18 | TGM2 | Transglutaminase 2 | 6.86E−04 |
| 19 | GCH1 | GTP cyclohydrolase 1 | 6.66E−04 |
| 20 | UCHL1 | Ubiquitin carboxyl-terminal esterase L1 | 6.60E−04 |

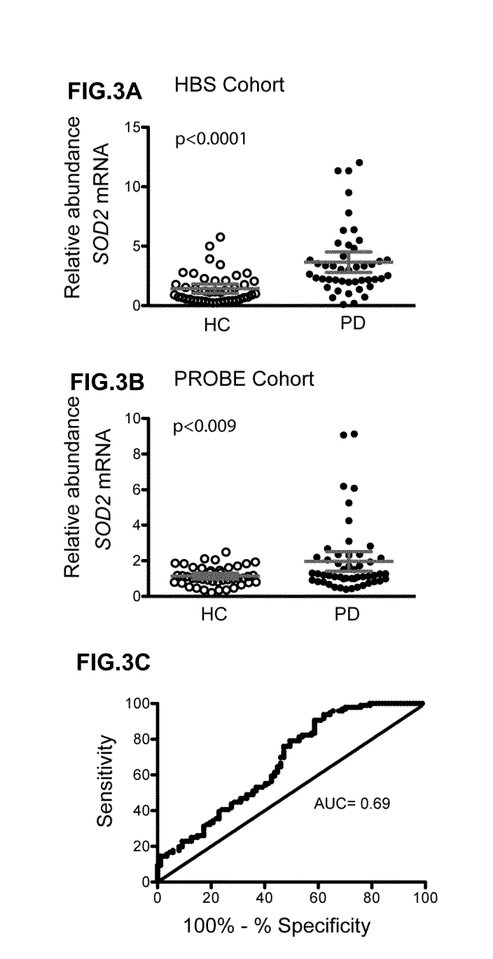

In order to validate the results obtained from the network analysis, the most highly ranked gene, SOD2, was evaluated as a potential biomarker for PD. Relative abundance of SOD2 mRNA was measured in whole blood of PD patients compared to HC from samples obtained from two independent clinical trials, HBS and PROBE. Quantitative PCR assays revealed that SOD2 mRNA is significantly upregulated in blood of PD patients compared to HC in both cohorts of study participants, although significant overlap in expression levels was observed between PD and controls (FIGS. 3A and 3B). To evaluate the diagnostic accuracy of SOD2 in distinguishing PD patients from HC, ROC curve analysis was performed. As shown in FIG. 3C, the AUC value for SOD2 was 0.69.

Pearson correlation analysis demonstrated that relative abundance of SOD2 was independent of other covariates including age (r=−0.13, p=0.40), and sex (r=−0.03, p=0.79) in both cohorts of patients and BMI (r=0.18, p=0.21) in the HBS cohort. Correlation analysis of SOD2 mRNA expression and Hoehn and Yahr stage was not significant (r=0.04, p=0.73). Correlation with medication was not determined since most of the patients with PD were medicated with several drugs and the number of untreated patients was too small to reliably detect a significant change.

### Discussion

This study integrated data from publicly available databases that includes a wide range of experimental designs including, but not limited to, pharmacogenomics, toxicogenomics, and other experiments in addition to GWAS, and identified 84 genes shared between PD and T2DM by interrogating several disease-gene databases. Biological and functional analysis of these genes identified shared dysregulated pathways including nitric oxide biosynthesis, regulation of glucose, lipid and carbohydrate metabolism, insulin secretion and inflammation. Shared genes between both diseases were prioritized using a RWR within the human FLN. Highly ranked genes were representative of the most significant dysregulated pathways. For example, AKT1, IGF1 and TP53 are involved in insulin signaling and glucose homeostasis. In this regard, dysregulation of glucose metabolism was identified as an early event in sporadic PD and it has been hypothesized that alpha synuclein (SNCA) may play a role in this process. In addition, genes associated with inflammation including TNF and IL6 are among the most highly ranked genes. In this context, neuroinflammation is associated with the pathophysiology of PD. HNF4A was also among the top 10 genes in the prioritized list. Interestingly, HNF4A may also play a role in intestinal lipid metabolism, oxidative stress and inflammation, processes that are implicated in both chronic diseases. Collectively, these findings are consistent with previous studies that highlight the significant convergence of dysregulated pathways in PD and T2DM (Santiago & Potashkin J A (2013) Shared dysregulated pathways lead to Parkinson's disease and diabetes. Trends in Molecular Medicine 19: 176-186; Santiago & Potashkin J A (2013) Integrative network analysis unveils convergent molecular pathways in Parkinson's disease and diabetes. PloS One 8: e83940; and Menon & Farina C (2011) Shared molecular and functional frameworks among five complex human disorders: a comparative study on interactomes linked to susceptibility genes. PloS One 6: e18660).

A highly ranked gene, SOD2, was further evaluated in blood of patients with PD from two independent cohorts of study participants. Relative abundance of SOD2 was upregulated in blood of PD patients compared to healthy individuals. SOD2 is a mitochondrial enzyme that protects against oxidative stress by converting superoxide radicals to molecular oxygen and hydrogen peroxide. Given its antioxidant capacity, it has been implicated in the pathogenesis of PD. For example, inactivation of SOD2 increases mitochondrial ROS production in in vitro models of PD. Moreover, SOD2 protein levels are increased in the frontal cortex of PD patients. In the context of diabetes, increased levels of SOD2 mRNA have been found in skeletal muscle of patients with T2DM (Reyna et al. (2008) Elevated toll-like receptor 4 expression and signaling in muscle from insulin-resistant subjects. Diabetes 57: 2595-2602). In addition, SOD2 has been associated to be involved in inflammation, insulin signaling and glucose metabolism, and lipid metabolism and peroxidation, processes that were identified dysregulated in the network analysis.

Recently, drugs to treat diabetic patients, metformin-sulfonylurea and exenatide have shown promise in PD patients. In fact, improvement of motor and cognitive functions persists one year after the treatment with exenatide. Interestingly, diabetic drugs are known to interact with SOD2. For example, metformin treatment results in an increased expression of SOD2 mRNA in human endothelial cells. Troglitazone treatment, another anti-diabetic and anti-inflammatory drug, results in decreased expression of SOD2 mRNAs in cellular models. In addition, gliclazide treatment, an oral sulfonylurea hypoglycemic agent, results in decreased protein expression of SOD2, and rosiglitazone, an insulin sensitizer, increased SOD2 protein expression in retinal cells from mice. Based on these observations, expression of SOD2 in blood may be useful to evaluate the therapeutic effect of anti-diabetic drugs in PD patients.

This study has several strengths and limitations. Biomarkers obtained from microarray studies may be data set specific and not indicative of the underlying disease pathology. In this context, the integrated network approach here provides a framework to identify and prioritize PD biomarkers involved in common dysregulated pathways. Another strength is the replication of this biomarker in two independent cohorts of patients. However, there are several limitations and potential confounding factors. For example, although we have found that GAPDH mRNA expression in blood is stable in previous studies, replication of this biomarker using several reference genes for normalization is desirable. In addition, differences in blood counts and PD medications may bias gene expression results. Thus, evaluation of SOD2 mRNA in drug-naïve PD patients and in a large well-characterized prospective study will be important to determine its clinical utility.

In summary, this study demonstrates that integration of shared molecular networks provides a useful framework to prioritize candidate biomarkers in a biologically relevant context. Remarkably, we demonstrate that expression of a highly ranked gene identified within shared dysregulated pathways can be used as diagnostic marker for PD. Integrated network approaches will provide a better understanding of the underlying disease mechanism and facilitate the discovery of accurate biomarkers and therapeutic targets. In this regard, a network-based approach was useful to identify a neuroprotective agent, alvespimycin (17-DMAG), in PD. Although the prioritization method presented in this study has been evaluated in the specific case of PD-T2DM, other disease-disease associations may be studied following this protocol. For instance, the construction of shared genes and protein networks has facilitated the understanding of other disease-disease associations such as asthma and tuberculosis and artherosclerosis-induced ocular diseases. Thus, network analysis of disease comorbidities may reveal novel diagnostic biomarkers and therapeutic strategies.

1. A method for diagnosing, prognosing or monitoring Parkinson's Disease (PD) in a human subject, the method comprising:
(a) obtaining a blood sample from a human subject suspected of having PD;
(b) determining the expression level of at least one gene in the blood sample from the human subject suspected of having PD, wherein the at least one gene is selected from: SOD2, MT-ND1, TNF, IFNG, TP53, IL6, AKT1, HNF4A, HMOX1, FAS, APP, CYP17A1, IGF1, PTGS2, SOD1, BDNF, NOS2, TGM2, GCH1, UCHL1, IL1B, HNF1A, APOE, IGF2, CYP1A1, PPARG, SLC18A2, CD14, PINK1, INS, PARP1, NFKB1, SLC2A4, IDE, DRD2, GAD2, SORBS1, CP, TH, TSC2, PON1, E2F1, CXCR4, CDKN2A, KCNJ2, PPARGC1A, HGF, OPRM1, TF, ACE, CADM1, NQO1, GAD1, GH1, HFE, CXCL12, ABCB1, MAOB, BTG1, ABCC8, PDX1, ADH1C, CCL5, TCF7L2, ATF6, GPX1, CCL2, VDR, MTHFR, IL8, KIF11, MMP16, GSTM1, HP, NPPB, SEMA6A, NCAM2, SERPINB1, NAT2, MBNL1, PCDH18, OLFM4, RBMS3 and TBC1D22A; and
(c) comparing the expression level of the at least one gene expressed in the blood sample to the expression level of the at least one gene in a non-PD, healthy control sample, whereby the increased or decreased expression level of the at least one gene expressed in the blood sample from the human subject suspected of having PD as compared to the non-PD sample is indicative of PD, thereby diagnosing the human subject as having PD.

2. The method of claim 1, wherein the at least one gene is SOD2 encoded by SEQ ID NO: 01.

3. The method of claim 1, wherein the expression level is determined by detecting messenger RNA of the at least one gene.

4. The method of claim 1, further comprising reverse transcription of the messenger RNA prior to detecting.

5. The method of claim 1, wherein determining the expression level of the at least one gene is by measuring a level of fluorescence by a sequence detection system following a quantitative, real-time polymerase chain reaction (PCR) assay.

6. The method of claim 1, further comprising determining a treatment regimen for the human subject.

7. A method of treating a human subject for Parkinson's Disease (PD), the method comprising:
(a) obtaining a diagnosis identifying a human subject as having PD, wherein the diagnosis was obtained by:
(i) obtaining a blood sample from a human subject suspected of having PD;
(ii) determining the expression level of at least one gene in the blood sample from the human subject suspected of having PD, wherein the at least one gene is selected from: SOD2, MT-ND1, TNF, IFNG, TP53, IL6, AKT1, HNF4A, HMOX1, FAS, APP, CYP17A1, IGF1, PTGS2, SOD1, BDNF, NOS2, TGM2, GCH1, UCHL1, IL1B, HNF1A, APOE, IGF2, CYP1A1, PPARG, SLC18A2, CD14, PINK1, INS, PARP1, NFKB1, SLC2A4, IDE, DRD2, GAD2, SORBS1, CP, TH, TSC2, PON1, E2F1, CXCR4, CDKN2A, KCNJ2, PPARGC1A, HGF, OPRM1, TF, ACE, CADM1, NQO1, GAD1, GH1, HFE, CXCL12, ABCB1, MAOB, BTG1, ABCC8, PDX1, ADH1C, CCL5, TCF7L2, ATF6, GPX1, CCL2, VDR, MTHFR, IL8, KIF11, MMP16, GSTM1, HP, NPPB, SEMA6A, NCAM2, SERPINB1, NAT2, MBNL1, PCDH18, OLFM4, RBMS3 and TBC1D22A; and
(iii) comparing the expression level of the at least one gene expressed in the blood sample to the expression level of the at least one gene expressed in a non-PD, healthy control sample, whereby the increased or decreased expression level of the at least one gene expressed in the blood sample from the human subject suspected of having PD as compared to the non-PD sample is indicative of PD, thereby diagnosing the human subject as having PD; and
(b) administering to the subject a PD treatment regimen.

8. The method of claim 7, wherein the at least one gene is SOD2 encoded by SEQ ID NO: 01.

9. The method of claim 7, wherein the expression level is determined by detecting messenger RNA of the at least one gene.

10. The method of claim 7, further comprising reverse transcription of the messenger RNA prior to detecting.

11. The method of claim 7, wherein determining the expression level of the at least one gene is by measuring a level of fluorescence by a sequence detection system following a quantitative, real-time polymerase chain reaction (PCR) assay.

12. A Parkinson's Disease (PD) diagnosis, prognosis or monitoring kit, consisting of a set of probes suitable for the detection and quantification of the nucleic acid expression of at least one gene selected from: SOD2, MTND1, TNF, IFNG, TP53, IL6, AKT1, HNF4A, HMOX1, FAS, APP, CYP17A1, IGF1, PTGS2, SOD1, BDNF, NOS2, TGM2, GCH1, UCHL1, IL1B, HNF1A, APOE, IGF2, CYP1A1, PPARG, SLC18A2, CD14, PINK1, INS, PARP1, NFKB1, SLC2A4, IDE, DRD2, GAD2, SORBS1, CP, TH, TSC2, PON1, E2F1, CXCR4, CDKN2A, KCNJ2, PPARGC1A, HGF, OPRM1, TF, ACE, CADM1, NQO1, GAD1, GH1, HFE, CXCL12, ABCB1, MAOB, BTG1, ABCC8, PDX1, ADH1C, CCL5, TCF7L2, ATF6, GPX1, CCL2, VDR, MTHFR, IL8, KIF11, MMP16, GSTM1, HP, NPPB, SEMA6A, NCAM2, SERPINB1, NAT2, MBNL1, PCDH18, OLFM4, RBMS3 and TBC1D22A.
